(19) United States
(12) Patent Application Publication
(10) Pub. No.: US 2024/0325107 A1
(43) Pub. Date: Oct. 3, 2024

(54) SYSTEM OF REAL-TIME DISPLAYING PROMPT IN SYNCHRONOUS DISPLAYED SURGICAL OPERATION VIDEO AND METHOD THEREOF

(71) Applicant: SMART SURGERY, Taipei (TW)

(72) Inventor: Yu-Chieh LEE, Taipei (TW)

(21) Appl. No.: 18/622,860

(22) Filed: Mar. 29, 2024

(30) Foreign Application Priority Data
Mar. 31, 2023 (TW) 112112567

(51) Int. Cl. A61B 90/00; A61B 34/10; G06F 3/01; G06V 10/44; G16H 30/40; H04N 21/81

(52) U.S. Cl. CPC A61B 90/361; A61B 34/10; A61B 90/37; G06F 3/013; G06F 3/017; G06V 10/44; G16H 30/40; H04N 21/816

(57) ABSTRACT

A system of real-time displaying prompt in synchronous displayed surgical operation video and a method thereof are disclosed. In the system, 2D surgical operation videos are synchronously captured from different viewing angles, and a target part is generated based on an obtained instruction message, a prompt corresponding to the target part in the two 2D surgical operation videos is generated, and the two 2D surgical operation videos are projected to left and right eyes of a viewer, respectively, to make the viewer watch a 3D surgical operation video, to effectively present depths of objects in a surgical operation video. Therefore, the effect of reducing surgical errors caused by doctors misjudging depths of objects in surgical operation videos can be achieved.

### CROSS-REFERENCE STATEMENT

The present application is based on, and claims priority from, TAIWAN Patent Application Serial Number 112112567, filed Mar. 31, 2023, the disclosure of which is hereby incorporated by reference herein in its entirety.

### BACKGROUND

### 1. Technique Field

The present invention is related to a real-time video prompting system, and particularly to a system of real-time displaying prompt in synchronous displayed surgical operation video and a method thereof.

### 2. RELATED ART

In recent years, with the popularization and vigorous development of medical technology, various related applications have sprung up, such as additional applications of surgical operation videos.

Generally, surgical operation videos serve as learning materials for physicians, students, or related practitioners, and even serve as surgery records to avoid medical disputes. To improve the learning effect, some manufacturers use image recognition technology combined with surgical operation videos to label the organs and tissues appearing in the surgical operation videos.

However, the above-mentioned surgical operation videos are mostly two-dimensional (2D) videos, or three-dimensional (3D) videos simulated by 2D videos, which are the videos usually captured from single eye (left eye or right eye) of chief surgeon. Therefore, the conventional surgical operation videos are unable to effectively present depths of objects (also called as depth of field), so a viewer needs to have a certain medical knowledge background to effectively understand and determine the depths of objects in the surgical operation video. When the viewer is an assistant surgeon in the same surgery, the assistant surgeon needs to have certain image surgery experience to accurately assist the chief surgeon and correctly complete the instructions given by the chief surgeon through watching the surgical operation videos. As a result, once the assistant surgeon is inexperienced, the possibility of errors in a surgery is increased.

According to above-mentioned contents, what is needed is to develop an improved solution to solve the conventional problem that the existing surgical operation videos with organ labels or tissue labels are unable to effectively display depths of objects.

### SUMMARY

An objective of the present invention is to disclose a system of real-time displaying prompt in synchronous displayed surgical operation video and a method thereof, to solve the conventional problem that the existing surgical operation videos with organ labels or tissue labels are unable to effectively display depths of objects.

In order to achieve the objective, the present invention discloses a system of real-time displaying prompt in synchronous displayed surgical operation video. The system includes an image capturing module, an image display module, and a processing module. The image capturing module is configured to synchronously capture two 2D surgical operation videos from different viewing angles during a surgery. The image display module includes a 3D display. The processing module is connected to the image capturing module and the image display module, and configured to execute the computer readable instructions to generate an image processing module, a message obtaining module, a target determining module, a position determining module, and a label generating module. The image processing module is configured to generate a naked eye 3D video corresponding to the 3D display in real time based on the two 2D surgical operation videos, to make the 3D display synchronously project the two 2D surgical operation videos to the left and right eyes of a viewer based on the naked eye 3D video, respectively, to make the viewer watch a 3D surgical operation video. The message obtaining module is configured to obtain an instruction message. The target determining module is configured to determine a target part related to the instruction message. The position determining module is configured to determine a label position of the target part in each of the two 2D surgical operation videos based on feature data of the target part. The label generating module is configured to generate a prompt corresponding to the target part in the two 2D surgical operation videos based on the label positions, to make the prompt be displayed in the 3D surgical operation video watched by the viewer.

In order to achieve the objective, the present invention discloses a method of real-time displaying prompt in synchronous displayed surgical operation video. The method includes steps of: during a surgery, synchronously capturing two two-dimensional (2D) surgical operation videos from different viewing angles, by the device; generating a naked eye three-dimensional (3D) video corresponding to a 3D display in real time, based on the two 2D surgical operation videos, by the device; using the 3D display to synchronously project the two 2D surgical operation videos to left and right eyes of a viewer based on the naked eye 3D video, respectively, to make the viewer watch a 3D surgical operation video, by the device; obtaining an instruction message, by the device; determining a target part related to the instruction message, by the device; determining a label position of the target part in each of the two 2D surgical operation videos based on feature data of the target part, by the device; generating a prompt corresponding to the target part in the two 2D surgical operation videos based on the label positions, to make the prompt be displayed in the 3D surgical operation video watched by the viewer, by the device.

According to the above-mentioned system and method of the present invention, the difference between the present invention and the conventional technology is that, in the present invention, the 2D surgical operation videos are synchronously captured from different viewing angles, and the target part is generated based on the obtained instruction message, the prompt corresponding to the target part in the two 2D surgical operation videos is generated, and the 3D display projects the two 2D surgical operation videos to left and right eyes of a viewer, respectively, to make the viewer watch the 3D surgical operation video, so as to solve the conventional problem and achieve the effect of reducing surgical errors caused by doctors misjudging depths of objects in surgical operation videos.

### BRIEF DESCRIPTION OF THE DRAWINGS

The structure, operating principle and effects of the present invention will be described in detail by way of various embodiments which are illustrated in the accompanying drawings.

FIG. 1A is a structural view of a system of real-time displaying prompt in synchronous displayed surgical operation video, according to the present invention.

FIG. 1B is a schematic view of functional modules of a system of real-time displaying prompt in synchronous displayed surgical operation video, according to the present invention.

FIG. 2A is a schematic view of multiple devices of an embodiment of the present invention.

FIG. 2B is a schematic view of multiple devices of another embodiment of the present invention.

FIG. 3A is a flowchart of a method of real-time displaying prompt in synchronous displayed surgical operation video, according to the present invention.

FIG. 3B is a flowchart of an operation of tracking a viewer's eye to adjust a projecting angle, according to the present invention.

FIG. 4A is a schematic view of a frame of a 2D surgical operation video, according to an embodiment of the present invention.

FIG. 4B is a schematic view of a screen having color block labels and text descriptions, according to an embodiment of the present invention.

### DETAILED DESCRIPTION OF THE PREFERRED EMBODIMENTS

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims.

These embodiments are provided so that this disclosure is thorough and complete, and fully conveys the inventive concept to those skilled in the art. Regarding the drawings, the relative proportions, and ratios of elements in the drawings may be exaggerated or diminished in size for the sake of clarity and convenience. Such arbitrary proportions are only illustrative and not limiting in any way. The same reference numbers are used in the drawings and description to refer to the same or like parts. As used herein, the singular forms “a”, “an” and “the” are intended to include the plural forms as well, unless the context clearly indicates otherwise.

It is to be acknowledged that, although the terms ‘first,’ ‘second,’ ‘third,’ and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the words “comprise” and “include,” and variations such as “comprises,” “comprising,” “includes,” or “including,” will be acknowledged to imply the inclusion of stated elements but not the exclusion of any other elements.

The solution of the present invention is able to synchronously display a surgical operation video through a naked eye 3D image, and to indicate a target part related to an instruction message during a video displaying process. The present invention, including the form of system and method, can be applied to single device, or multiple devices connected to each other. The device mentioned in the present invention can be implemented by computing apparatus. In an embodiment, the video is an image stream.

The computing apparatus mentioned in the present invention can include, but not limited to, one or more processing module, one or more memory module, and a bus connected to different hardware components including the memory module and the processing module. Through the multiple hardware components, the computing apparatus can load and execute the operating system, so that the operating system runs on the computing apparatus and executes software or programs. In addition, the computing apparatus can include an outer shell, and the above-mentioned hardware component are disposed in the outer shell.

The bus mentioned in the present invention can include at least one type of bus, for example, the bus can include at least one of a data bus, an address bus, a control bus, an expansion bus, and a local bus. The bus of a computation device can include, but not limited to, a parallel bus such as an industry standard architecture (ISA) bus, a peripheral component interconnect (PCI) bus, a video electronics standards association (VESA) local bus, or a serial bus such as a USB, or a PCI express (PCI-E/PCIe) bus.

The processing module of the computing apparatus is coupled with the bus. The processing module includes a register group or a register space. The register group or the register space can be completely set on the processing chip of the processing module, or can be all or partially set outside the processing chip and is coupled to the processing chip through dedicated electrical connection and/or a bus. The processing module can be a central processing unit, a microprocessor, or any suitable processing component. If the computing apparatus is a multi-processor apparatus, that is, the computing apparatus includes processing modules, and the processing modules can be all the same or similar, and coupled and communicated with each other through a bus. The processing module can interpret a computer instruction or a series of multiple computer instructions to perform specific operations or operations, such as mathematical operations, logical operations, data comparison, data copy/moving, so as to drive other hardware component, execute the operating system, or execute various programs and/or module in the computing apparatus. The computer instructions can include assembly language instructions, instruction set architecture instructions, machine instructions, machine-related instructions, microinstructions, firmware instructions, or source code or object code written in one or more programming languages. The instructions can be executed entirely on a single computing apparatus, partially on a single computing apparatus, or partially on one computing apparatus and partially on another interconnected computing apparatus. The above-mentioned programming language can be, for example, object-oriented languages such as Common Lisp, Python, C++, Objective-C, Smalltalk, Delphi, Java, Swift, C#, Perl, Ruby, as well as procedural languages like C or similar languages.

The computing apparatus usually also includes one or more chipsets. The processing module of the computing apparatus can be coupled to the chipset, or electrically connected to the chipset through the bus. The chipset includes one or more integrated circuits (IC) including a memory controller and a peripheral input/output (I/O) controller, that is, the memory controller and the peripheral input/output controller can be implemented by one integrated circuit, or implemented by two or more integrated circuits. Chipsets usually provide I/O and memory management functions, and multiple general-purpose and/or dedicated-purpose registers, timers. The above-mentioned general-purpose and/or dedicated-purpose registers and timers can be coupled to or electrically connected to one or more processing modules to the chipset for being accessed or used.

The processing module of the computing apparatus can also access the data stored in the memory module and mass storage area installed on the computing apparatus through the memory controller. The above-mentioned memory modules include any type of volatile memory and/or non-volatile memory (NVRAM), such as Static Random Access Memory (SRAM), Dynamic Random Access Memory (DRAM), Read-Only Memory (ROM), or Flash memory. The above-mentioned mass storage area can include any type of storage device or storage medium, such as hard disk drives, optical discs, flash drives, memory cards, and solid state disks (SSD), or any other storage device. In other words, the memory controller can access data stored in static random access memory, dynamic random access memory, flash memory, hard disk drives, and solid state drives.

The processing module of the computing apparatus can also connect and communicate with peripheral devices and interfaces including peripheral output devices, peripheral input devices, communication interfaces, or data/signal receivers through the peripheral I/O controller and the peripheral I/O bus. The peripheral input device can be any type of input device, such as a keyboard, mouse, trackball, touchpad, or joystick. The peripheral output device can be any type of output device, such as a display, or a printer; the peripheral input device and the peripheral output device can also be the same device such as a touch screen. The communication interface can include a wireless communication interface and/or a wired communication interface. The wireless communication interface can include the interface capable of supporting wireless local area networks (such as Wi-Fi, Zigbee, etc.), Bluetooth, infrared, and near-field communication (NFC), 3G/4G/5G and other mobile communication network (cellular network) or other wireless data transmission protocol; the wired communication interface can be an Ethernet device, a DSL modem, a cable modem, an asynchronous transfer mode (ATM) devices, or optical fiber communication interfaces and/or components. The data/signal receiver can include a GPS receiver or physiological signal receiver. The physiological signals received by the physiological signal receiver include, but are not limited to, heartbeat, blood oxygen levels, and so on. The processing module can periodically poll various peripheral devices and interfaces, so that the computing apparatus can input and output data through various peripheral devices and interfaces, and can also communicate with another computing apparatus having the above-mentioned hardware components.

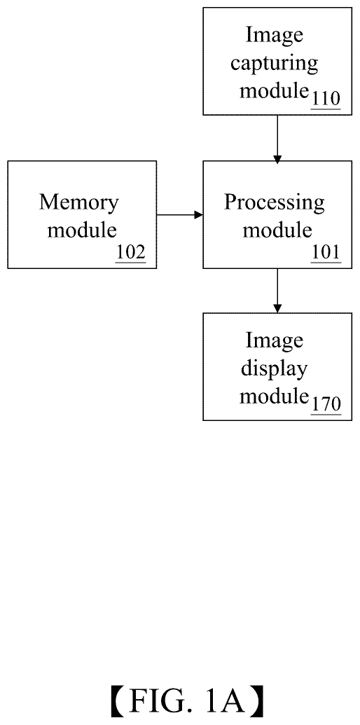

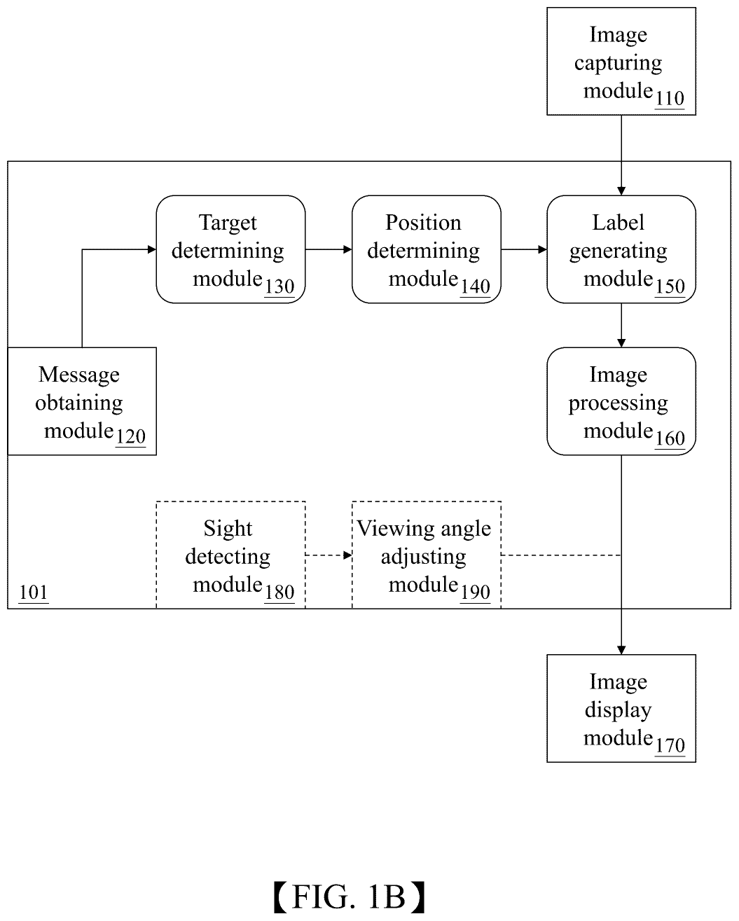

The system of the present invention will be illustrated in the following paragraphs with reference to FIG. 1. As shown in FIG. 1A, the system includes a processing module 101, an image capturing module 110, and an image display module 170.

Please refer to FIG. 1B. FIG. 1B is a schematic view of functional modules of a system of real-time displaying prompt in synchronous displayed surgical operation video, according to the present invention. The processing module 101 includes a message obtaining module 120, a target determining module 130, a position determining module 140, a label generating module 150, an image processing module 160; optionally, the processing module 101 can include a sight detecting module 180, and a viewing angle adjusting module 190.

In an embodiment, the processing module 101 executes one computer readable instruction or a series of (such as multiple, a set of, or sets of) computer readable instructions stored in the memory module 102, so that the above-mentioned computer instruction are executed to generate the message obtaining module 120, the target determining module 130, the position determining module 140, the label generating module 150, the image processing module 160, the sight detecting module 180, and the viewing angle adjusting module 190; in another embodiment, the processing module 101 can include physical bodies of the above-mentioned modules 120˜160, and 180˜190; in other words, the above-mentioned modules 120˜160 and 180˜190 are implemented by hardware components such as physical circuits or electronic components or chips, that is, the processing module 101 is a general term of circuits or electronic components or chips for implementing the above-mentioned modules 120˜160, and 180˜190. The electronic components can include, but not limited to complex programmable logic device, (CPLD); the above-mentioned chip can be, for example, application specific integrated circuit (ASIC), system on a chip (SoC), field programmable gate array (FPGA); however, the present invention is not limited to above-mentioned examples.

During a surgery, the image capturing module 110 synchronously captures two 2D surgical operation videos from different viewing angles. Generally speaking, the image capturing module 110 can capture two or more 2D surgical operation videos, and each 2D surgical operation video is captured from different viewing angle. For example, the image capturing module 110 can be an image capturing device having two or more camera lenses, and the image capturing device can be controlled to synchronously capture the 2D surgical operation videos from different viewing angles, that is, the 2D surgical operation video is an image stream having different viewing angle; however, the present invention is not limited to above-mentioned examples.

The message obtaining module 120 is configured to obtain an instruction message. The instruction message obtained by the message obtaining module 120 can include, but not limited to, voice, gesture, or screen operation. For example, the message obtaining module 120 receives the voice message (is also called as instructional voice herein) outputted from a chief surgeon or an instructing doctor in the 2D surgical operation video of the surgery, to obtain the instruction message, or the message obtaining module 120 analyzes the 2D surgical operation video obtained by the image capturing module 110 to detect a gesture motion (is also called as instructional gesture herein) of the chief surgeon or the instructing doctor within a surgical range of the surgery, to obtain the instruction message, or the message obtaining module 120 generates the instruction message corresponding to an instructional operation of the instructing doctor in the 2D surgical operation video projected by the image display module 170. The above-mentioned instructional operation can include, but not limited to, an operation of clicking or circling a specific object in the 2D surgical operation video.

The target determining module 130 is configured to determine the target part related to the instruction message obtained by the message obtaining module 120. When the message obtaining module 120 obtains the instructional voice, the target determining module 130 determines whether the instructional voice includes an organ or tissue or instrument related to the surgery, for example, the target determining module 130 can determine whether the instructional voice includes a speech signal having high-enough matching degree or similarity with an organ name, a tissue name, or an instrument name related to the surgery, or the target determining module 130 can perform a speech recognition on the instructional voice to obtain an instruction content of the instructional voice, and determine whether the instruction content has text of an organ name, a tissue name or an instrument name related to the surgery. If the determination result is false, the target determining module 130 determines that the instructional voice does not have related target part; if the determination result is true, the target determining module 130 uses the organ or tissue or instrument in the instruction content, as the target part. When the message obtaining module 120 obtains the instructional gesture, the target determining module 130 performs a feature analysis on the instructional gesture, and determine the related target part based on the analyzed gesture feature, for example, the target determining module 130 can select the feature (from prebuilt feature data) matching the gesture feature, and use the organ or tissue or instrument corresponding to the selected feature as the target part, or the target determining module 130 can determine the organ or tissue or instrument pointed or circled by the instructional gesture based on the gesture feature of the instructional gesture, and use the organ or tissue or instrument pointed or circled by the instructional gesture as the target part. When the message obtaining module 120 obtains the instructional operation, the target determining module 130 determines the position of the instructional operation occurred on the monitor displaying the naked eye 3D video, and uses the organ, or tissue or instrument displayed at the position on the monitor when the instructional operation occurs, as the target part. However, the manner used by the target determining module 130 to determine the target part is not limited to above-mentioned example.

The position determining module 140 determines a label position of the target part in the two or more 2D surgical operation videos captured by the image capturing module 110, based on the target part determined by the target determining module 130. For example, the position determining module 140 perform feature extraction on each frame of the 2D surgical operation video, to obtain the image feature; if necessary, the position determining module 140 can perform image analysis process (such as the grayscale conversion and/or edge detection) on the frame, and the position determining module 140 determines whether the obtained image feature includes a part matching the feature data of the target part. For example, the position determining module 140 can determine whether the similarity between the image feature and the feature data is higher than a certain value, if no, it indicates that the frame does not have the target part; if yes, it indicates that the frame has the target part. The position determining module 140 determines and obtains a range of the target part in the frame to generate the part message, based on a portion of the image feature matching the feature data of the target part. When the target determining module 130 determines the target part based on the instructional gesture at surgery site or based on the instructional operation in the 2D surgical operation video, the position determining module 140 performs image analysis process (such as grayscale conversion and/or edge detection) on each frame of the 2D surgical operation video, to determine the range of the target part in the 2D surgical operation video, obtains the image feature within the determined range of the target part, and determines whether the obtained image feature matches the feature data of the prebuilt known organ or tissue or instrument, if yes, the position determining module 140 generates the part message based on the image feature within the range of the target part, and the organ or tissue or instrument corresponding to the matching feature data. Generally speaking, the part message can record the time point or serial number of the frame of the 2D surgical operation video, and record an outline of the target part on the frame. In an embodiment, the part message can record the outline of the target part through multiple connected line sections (such as coordinates of origin point and end point) or vectors (such as a coordinate, a direction, and a length of origin point. However, the present invention is not limited to above-mentioned examples. The feature data of the target part is prebuilt through, for example, neural network (ANN) and/or deep learning. For example, a large number of images of organs, tissues and instruments are used as training data to generate the feature data.

The label generating module 150 is configured to generate a prompt corresponding to the target part determined by the target determining module 130 in the two or more 2D surgical operation videos captured by the image capturing module 110, based on the label position determined by the position determining module 140, to make the generated prompt be displayed in the 3D surgical operation video watched by the viewer.

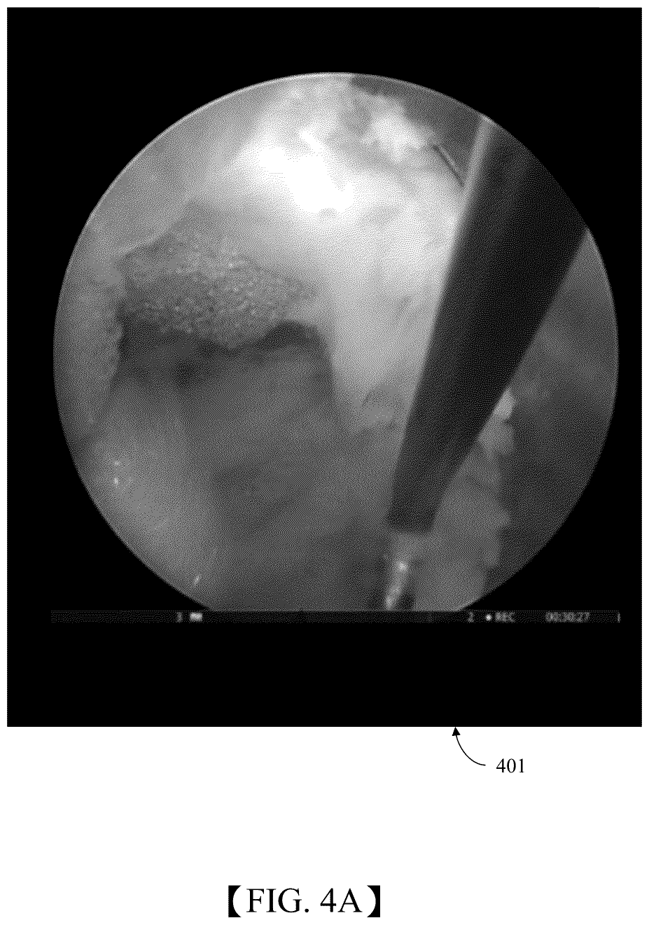

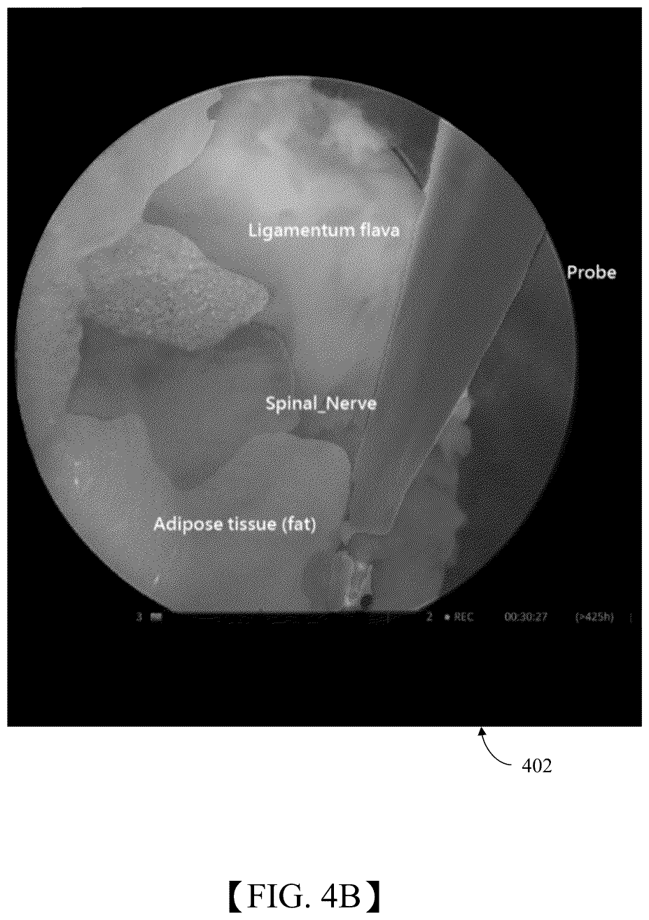

The prompt generated by the label generating module 150 can be a color block label or a text description; in more detail, the label generating module 150 can generate the color block label matching the label position determined by the position determining module 140 in the 2D surgical operation video captured by the image capturing module 110, so that the target part determined by the target determining module 130 can be indicated in the 2D surgical operation video captured by the image capturing module 110, for example, the label generating module 150 can generate the color block label on the outline on the frame based on the target part recorded in the part message generated by the position determining module 140. However, the method used by the label generating module 150 to generate the color block label is not limited to above-mentioned example, and any method capable of indicate a specific area based on the line sections or vectors in an image can be used in the present invention. The label generating module 150 can also generate a text description indicating the label position determined by the position determining module 140 in the one or more 2D surgical operation video captured by the image capturing module 110. For example, as shown in FIG. 4A, when a frame 401 of the 2D surgical operation video has organs and tissues such as ligamentum flava, spinal nerve or adipose tissue, and instruments such as a probe, the label generating module 150 generates the color block label and/or the text description corresponding to the organs, the tissues, and the instruments and in the frame 401, such as a screen 402 shown in FIG. 4B. It is worth mentioning that the text description can contain one or more languages, for example, both English and Chinese can be used in the text description.

It is to be noted that, when the label generating module 150 generates the text description in the 2D surgical operation videos, the label generating module 150 calculates a superposition position of the text description in each of the 2D surgical operation videos based on capturing angles of the 2D surgical operation videos captured by the image capturing module 110, so that the text descriptions can be fully overlaid in the 3D surgical operation video watched by the viewer, to prevent the text description from being not clearly displayed because the text description is not fully overlaid in the 3D surgical operation video watched by the viewer.

The image processing module 160 generates the naked eye 3D video corresponding to the 3D display of the image display module 170 in real time, based on the two or more 2D surgical operation videos captured by the image capturing module 110. For example, when the 3D display is a multiplexed-2D-type 3D display, the image processing module 160 combines the synchronous frames in each of the 2D surgical operation video to generate the naked eye 3D video, in more details, the image processing module 160 arranges vertical pixels (also called pixel column) of the synchronous frame in the 2D surgical operation video in sequential order; for example, when there are N 2D surgical operation videos, and each frame has M pixel columns, the pixel columns of each of the frames of the naked eye 3D video are the first pixel column of the synchronous first frame, the first pixel column of the synchronous second frame, . . . , the first pixel column of the synchronous N-th frame, the second pixel column of the first frame, the second pixel column of the second frame, . . . , the second pixel column of the N-th frame, . . . , the M-th pixel column of the first frame, the M-th pixel column of the second frame, . . . , the M-th pixel column of the N-th frame, in sequential order. Alternatively, the image processing module 160 can extract relative positions (or capturing angles) of the image capturing devices capturing the 2D surgical operation videos and combine the 2D surgical operation videos in sequential order to generate the naked eye 3D video. When there are N 2D surgical operation videos and each frame has M pixel columns and the synchronous frames of the 2D surgical operation videos are arranged in the horizontal direction in the naked eye 3D video, the first˜M-th pixel columns are the first frame, the (M+1)-th˜M-th pixel columns are the second frame, . . . (N−1)*M-th˜N*M-th pixel columns are N-th frame. However, the method used by the image processing module 160 to generate the naked eye 3D video is not limited to above-mentioned example.

The image display module 170 includes the 3D display, and is configured to control the 3D display thereof to make the 3D display synchronously project the two 2D surgical operation videos in different viewing angles to the left and right eyes of an viewer based on each frame of the naked eye 3D video, respectively, to make the viewer's brain merge the 2D surgical operation videos watched by the left and right eyes as the 3D surgical operation video. The 3D display can be multiplexed-2D-type display, such as a 3D display using parallax barriers or lenticular lenses; however, the present invention is not limited to above-mentioned examples, and a 3D display using a directional backlight or an E-holography 3D display can be used as the 3D display of the present invention.

The sight detecting module 180 detects dual-eye dynamics and a head movement of the viewer, to determine a watch sight of the viewer. For example, the sight detecting module 180 can include an image capturing unit to capture a face image of the viewer, and perform an image analysis process (such as edge detection, feature capture and feature analysis) on the captured face image, to determine positions of the viewer's head, eyes and pupils and then determine the viewer's watch sight through existing eye tracking solution based on the determined positions of the head, the eyes and pupils.

The viewing angle adjusting module 190 can adjust the viewing angles of the two 2D surgical operation videos projected by the 3D display of the image display module 170 based on the watch sight determined by the sight detecting module 180; for example, the viewing angle adjusting module 190 can adjust an angle (such as an elevation/depression angle) of the 3D display in the vertical direction and the angle of the 3D display in the horizontal direction, to make the 3D display project the 2D surgical operation video to the viewer by the optimal viewing angle, so that the viewer can watch the 2D surgical operation video displayed on the 3D display by the optimal angle.

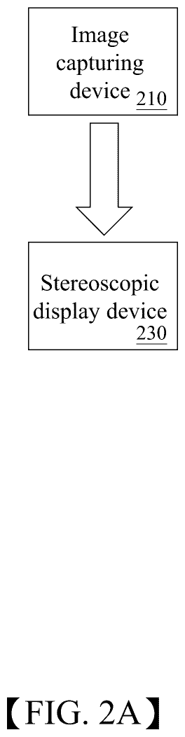

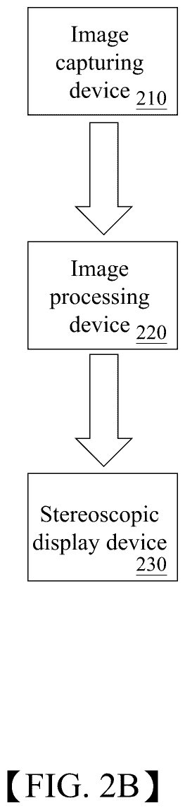

It is to be noted that the above-mentioned modules can be disposed in the same device, or distributed in different devices. As shown in FIG. 2A, the image capturing module 110 (and the message obtaining module 120) can be disposed in the image capturing device 210, and the remaining modules can be disposed on the stereoscopic display device 230. In another embodiment, as shown in FIG. 2B, the message obtaining module 120, the target determining module 130, the position determining module 140, the label generating module 150 and the image processing module 160 are separated from the stereoscopic display device 230 and disposed in the image processing device 220; however, the present invention is not limited to above-mentioned examples. The image capturing device 210 can transmit the 2D surgical operation videos captured by the image capturing module 110 to the label generating module 150 and/or the image processing module 160 of the stereoscopic display device 230 or the image processing device 220 through a wired or wireless manner, and the image processing module 160 of the image processing device 220 can transmit the naked eye 3D video to the image display module 170 of the stereoscopic display device 230 through a wired or wireless manner.

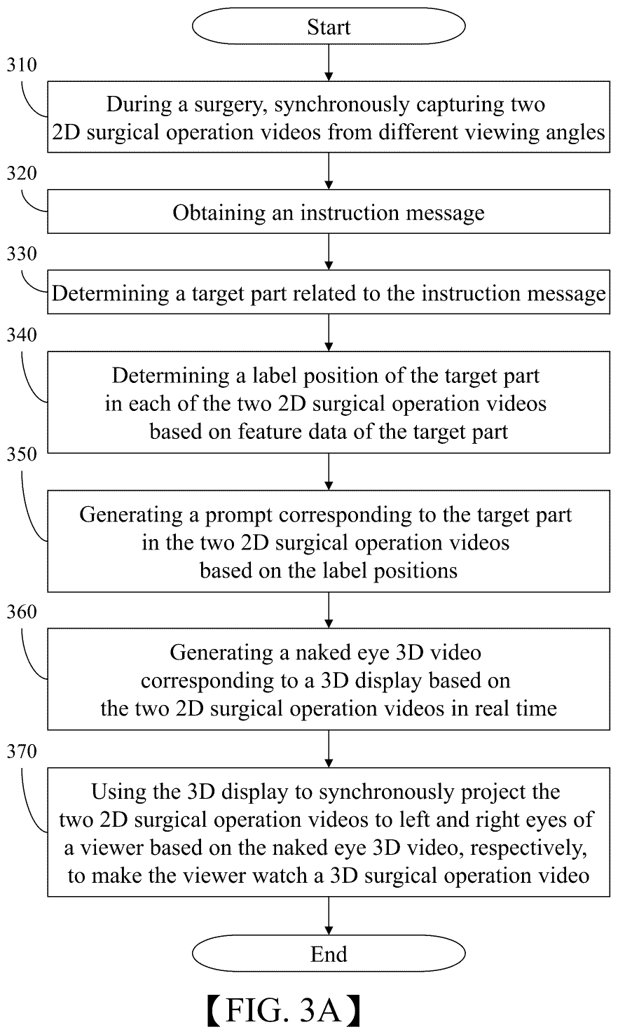

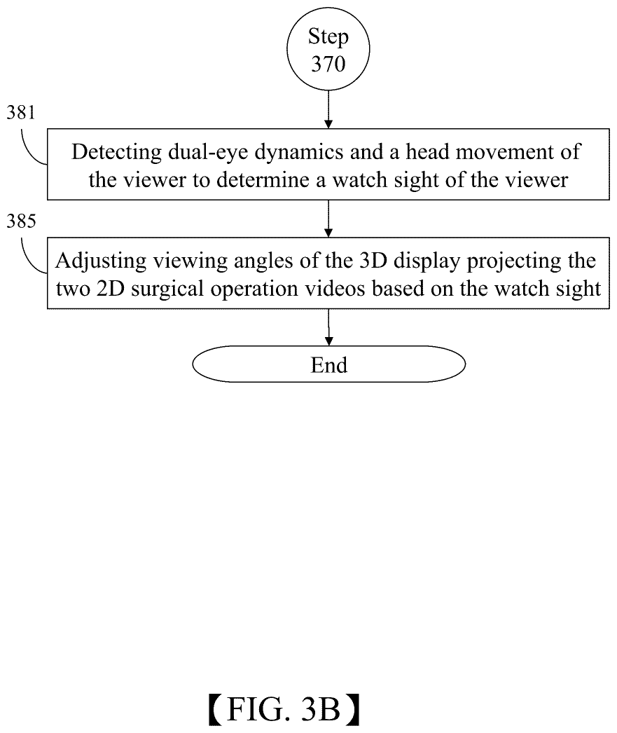

The operations of the system and the method of the present invention will be illustrated with reference to an embodiment, and please refer to FIG. 3A. FIG. 3A is a flowchart of a method of real-time displaying prompt in synchronous displayed surgical operation video, according to the present invention. In this embodiment, the present invention uses the image capturing device and the stereoscopic display device, and the stereoscopic display device is a display device of a surgery system; however, the present invention is not limited to above-mentioned examples.

During a surgery, the image capturing module 110 of the image capturing device synchronously captures the 2D surgical operation videos from different viewing angles (step 310). In this embodiment, the image capturing module 110 includes two image capturing devices disposed horizontally, the image capturing module 110 can control the two image capturing devices to synchronously capture the 2D surgical operation videos, and transmit the captured 2D surgical operation videos to the stereoscopic display device in streaming manner through a wireless transmission manner. The image capturing device is mounted around the surgery table, or is worn by the chief surgeon.

After the stereoscopic display device receives the two 2D surgical operation videos transmitted from the image capturing device, the image processing module 160 of the stereoscopic display device generates the naked eye 3D video corresponding to the 3D display of the image display module 170 of the stereoscopic display device based on the two received 2D surgical operation video (step 360), and the image display module 170 controls the 3D display to project different 2D surgical operation videos to the viewer's eyes by two different viewing angles, respectively, based on the naked eye 3D video, to make the viewer's brain merge the 2D surgical operation videos watched by the left and right eyes as the 3D surgical operation video (step 370). In this embodiment, the 3D display uses parallax barriers or lenticular lenses to project different 2D surgical operation videos to the assistant surgeon (the viewer), and the image processing module 160 can generate the naked eye 3D video in which the pixel columns of two 2D surgical operation videos are staggered in arrangement.

During the process where the image capturing module 110 of the image capturing device captures the two 2D surgical operation videos, the image processing module 160 of the stereoscopic display device generates the naked eye 3D video, the image display module 170 of the stereoscopic display device displays the naked eye 3D video based on the two 2D surgical operation videos, the message obtaining module 120 of the stereoscopic display device can obtain the instruction message (step 320). In this embodiment, the message obtaining module 120 can obtain an instructional voice through the multiple speech capturing unit when the chief surgeon speaks instruction, or obtain an instructional gesture through the multiple image capturing units when the chief surgeon makes a specific gesture.

After the message obtaining module 120 of the stereoscopic display device obtains the instruction message (step 320), the target determining module 130 of the stereoscopic display device can determine the target part related to the instruction message obtained by the message obtaining module 120 (step 330). In this embodiment, the target determining module 130 performs a speech recognition on the instructional voice to determine the target part such has the organ, tissue, or instrument related to the instructional voice, or performs a feature determination on the instructional gesture to obtain the target part such as an organ, a tissue, or an instrument.

After the target determining module 130 of the stereoscopic display device determines the target part, the position determining module 140 of the stereoscopic display device determines the label position of the target part in the two 2D surgical operation videos received by the stereoscopic display device based on the feature data of the target part determined by the target determining module 130 (step 340). In this embodiment, the position determining module 140 reads the prebuilt feature data of the target part, and perform feature extraction on each frame of the two 2D surgical operation videos to determine whether the image feature in the frame of the 2D surgical operation video matches the feature data of the target part, if yes, the position determining module 140 obtains the position of the target part in the frame of the 2D surgical operation video based on the image feature matching the feature data of the target part, and the position determining module 140 uses the obtained position as the label position.

After the position determining module 140 of the stereoscopic display device determines the label position of the target part in the 2D surgical operation video, the label generating module 150 generates a prompt corresponding to the target part in the frame of the 2D surgical operation video based on the label position determined by the position determining module 140 (step 350). In this embodiment, the label generating module 150 can generate a color block label at the label position on each of the frames of two 2D surgical operation videos, or generate a text description at the label position (indicating the target part) on the frame of at least one of the 2D surgical operation videos. When the label generating module 150 generates the text descriptions on the frames of both of the two 2D surgical operation videos, the label generating module 150 can determine the horizontal superposition positions of the text description on the frames of both of the 2D surgical operation videos projected to different eyes, based on a preset distance between the two image capturing devices of the image capturing module 110 of the image capturing device, and determine the same vertical superposition positions of the text description of the synchronous frames of both of the 2D surgical operation videos, so that the label generating module 150 can superposition the text description at the determined horizontal superposition positions and vertical superposition position on each frame.

After the label generating module 150 of the stereoscopic display device generates the prompt corresponding to the target part based on the label positions on the frames of the 2D surgical operation videos (step 350), the image processing module 160 of the stereoscopic display device generates the naked eye 3D video corresponding to the 3D display of the image display module 170 of the stereoscopic display device based on the two 2D surgical operation videos added with the prompt (step 360), and the image display module 170 controls the 3D display to project different 2D surgical operation videos to different eyes of the assistant surgeon (the viewer) by the two different viewing angles based on the naked eye 3D video, to make the assistant surgeon's brain merge the 2D surgical operation videos having the prompt and watched by the left and right eyes into the 3D surgical operation video (step 370).

As a result, with the above-mentioned technical solution of the present invention, when the chief surgeon performs a surgery, a surgical range watched by the chief surgeon can be synchronously displayed on the stereoscopic screen in real time for the assistant surgeon to watch, and the target part instructed by the chief surgeon can be indicated on the stereoscopic screen.

In above-mentioned embodiment, as the process shown in FIG. 3B, when the image display module 170 controls the 3D display to project different 2D surgical operation videos to the viewer's different eyes by the two different viewing angles based on the naked eye 3D video to make the viewer's brain merge the 2D surgical operation videos having the prompt and watched by the left and right eyes into the 3D surgical operation video (step 370), a sight detecting module 180 of the stereoscopic display device can capture a still image including a head and eyes of the assistant surgeon (that is, the viewer) and detect dual-eye dynamics and a head movement of the assistant surgeon based on the captured still image, to determine a watch sight of the viewer (step 381); next, the viewing angle adjusting module 190 in the stereoscopic display device can adjust the viewing angle of the 3D display of the image display module 170 projecting the 2D surgical operation videos to the left and right eyes of the assistant surgeon (the viewer) based on the watch sight determined by the sight detecting module 180 (step 385), so that the 3D display can project the 2D surgical operation video to eyes of the assistant surgeon (the viewer) by the optimal angle, that is, the assistant surgeon (the viewer) brain can merge the clearest 3D surgical operation video.

According to above-mentioned contents, the difference between the present invention and the conventional technology is that, in the present invention, the 2D surgical operation videos are synchronously captured from different viewing angles, and the target part is generated based on the obtained instruction message, the prompt corresponding to the target part in the two 2D surgical operation videos is generated, and the 3D display projects the two 2D surgical operation videos to left and right eyes of a viewer, respectively, to make the viewer watch the 3D surgical operation video, so as to solve the conventional problem that the existing surgical operation videos with organ labels or tissue labels are unable to effectively display depths of objects, so as to achieve the effect of reducing surgical errors caused by doctors misjudging depths of objects in surgical operation videos.

Furthermore, the above-mentioned method of real-time displaying prompt in synchronous displayed surgical operation video can be implemented by hardware, software, or a combination thereof, and can be implemented in a computer system by a centralization manner, or by a distribution manner of different components distributed in several interconnect computer systems.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. A method of real-time displaying prompt in a synchronous displayed surgical operation video, applicable to a device, and the method comprising:
during a surgery, synchronously capturing two two-dimensional (2D) surgical operation videos from different viewing angles, by the device;
generating a naked eye three-dimensional (3D) video corresponding to a 3D display based on the two 2D surgical operation videos in real time, by the device;
using the 3D display to synchronously project the two 2D surgical operation videos to left and right eyes of a viewer based on the naked eye 3D video, respectively, to make the viewer watch a 3D surgical operation video, by the device;
obtaining an instruction message, by the device;
determining a target part related to the instruction message, by the device;
determining a label position of the target part in each of the two 2D surgical operation videos based on feature data of the target part, by the device; and
generating a prompt corresponding to the target part in the two 2D surgical operation videos based on the label positions, to make the prompt be displayed in the 3D surgical operation video watched by the viewer, by the device.

2. The method of real-time displaying prompt in synchronous displayed surgical operation video according to claim 1, wherein the step of generating the prompt corresponding to the target part in the two 2D surgical operation videos based on the label positions by the device comprises:
displaying a color block label or a text description for indicating the target part corresponding to the label positions in the two 2D surgical operation videos, by the device, wherein the target part comprises an organ, a tissue, or an instrument.

3. The method of real-time displaying prompt in synchronous displayed surgical operation video according to claim 1, wherein the step of obtaining an instruction message by the device comprises:
receiving an instructional voice or detecting an instructional gesture within a surgical range to obtain the instruction message, or generating the instruction message based on an instructional operation for the naked eye 3D video displayed on a screen, by the device.

4. The method of real-time displaying prompt in synchronous displayed surgical operation video according to claim 3, wherein the step of determining the target part related to the instruction message by the device comprises:
analyzing a content of the instructional voice, or determining a position of the instructional gesture or the instructional operation in the naked eye 3D video, to determine the target part, by the device.

5. The method of real-time displaying prompt in synchronous displayed surgical operation video according to claim 1, wherein step of synchronously capturing the two 2D surgical operation videos from different viewing angles by the device comprises:
using an image capturing device with dual camera lenses to capture the two 2D surgical operation videos, by the device.

6. The method of real-time displaying prompt in synchronous displayed surgical operation video according to claim 1, wherein step of projecting the two 2D surgical operation videos by the device comprises:
detecting dual-eye dynamics and a head movement of the viewer to determine a watch sight of the viewer, and adjusting viewing angles of the 3D display projecting the two 2D surgical operation videos based on the watch sight, by the device.

7. A system of real-time displaying prompt in synchronous displayed surgical operation video, applicable to a device or multiple devices connected to each other, and the system comprising,
an image capturing module, configured to synchronously capture two 2D surgical operation videos from different viewing angles during a surgery;
an image display module, comprising a 3D display; and
a processing module, connected to the image capturing module and the image display module, and configured to execute the computer readable instructions to generate:
an image processing module, configured to generate a naked eye 3D video corresponding to the 3D display based on the two 2D surgical operation videos in real time, to make the 3D display synchronously project the two 2D surgical operation videos to the left and right eyes of a viewer based on the naked eye 3D video, respectively, to make the viewer watch a 3D surgical operation video;
a message obtaining module, configured to obtain an instruction message;
a target determining module, configured to determine a target part related to the instruction message;
a position determining module, configured to determine a label position of the target part in each of the two 2D surgical operation videos based on feature data of the target part; and
a label generating module, configured to generate a prompt corresponding to the target part in the two 2D surgical operation videos based on the label positions, to make the prompt be displayed in the 3D surgical operation video watched by the viewer.

8. The system of real-time displaying prompt in synchronous displayed surgical operation video according to claim 7, wherein the label generating module displays a color block label or a text description for indicating the target part corresponding to the label positions in the two 2D surgical operation videos, to generate the prompt, wherein the target part comprises an organ, a tissue, or an instrument.

9. The system of real-time displaying prompt in synchronous displayed surgical operation video according to claim 7, wherein the message obtaining module receives an instructional voice or detects an instructional gesture within a surgical range to obtain the instruction message, or generate the instruction message based on an instructional operation for the naked eye 3D video displayed on a screen, and the target determining module analyzes a content of the instructional voice, or determines a position corresponding to the instructional gesture or the instructional operation in the naked eye 3D video, to determine the target part.

10. The system of real-time displaying prompt in synchronous displayed surgical operation video according to claim 7, wherein the image capturing module comprises an image capturing device with dual camera lenses to capture the two 2D surgical operation videos.

11. The system of real-time displaying prompt in synchronous displayed surgical operation video according to claim 7, wherein the processing module comprises a sight detecting module and a viewing angle adjusting module, the sight detecting module detects dual-eye dynamics and a head movement of the viewer to determine a watch sight of the viewer, and the viewing angle adjusting module adjusts viewing angles of the 3D display projecting the two 2D surgical operation videos based on the watch sight.
